(19) United States
(12) Patent Application Publication
(10) Pub. No.: US 2026/0102514 A1
(43) Pub. Date: Apr. 16, 2026

(54) CHOLINERGIC GENE THERAPY TO PRESERVE HEARING

(71) Applicant: THE JOHNS HOPKINS UNIVERSITY, Baltimore, MD (US)

(72) Inventors: Paul A. Fuchs, Baltimore, MD (US); Ana Belen Elgoyhen, Baltimore, MD (US); Hakim Hiel, Baltimore, MD (US)

(21) Appl. No.: 19/115,754

(22) PCT Filed: Sep. 27, 2023
(86) PCT No.: PCT/US2023/033849

§ 371 Date: Mar. 26, 2025

Related U.S. Application Data
(60) Provisional application No. 63/410,543, filed on Sep. 27, 2022.

(51) Int. Cl. A61K 48/00; A61K 9/00; A61K 38/17; A61K 45/06; A61P 27/16; C12N 15/86

(52) U.S. Cl. CPC A61K 48/005; A61K 9/0046; A61K 38/1787; A61K 45/06; A61K 48/0075; A61P 27/16; C12N 15/86; C12N 2750/14143

(57) ABSTRACT

Provided herein are vectors including a polynucleotide encoding an α9L9′T protein, pharmaceutical compositions including the vector including a polynucleotide encoding an α9L9′T protein, and methods of use thereof. The methods of use include methods of preventing hearing loss, methods of protecting against or treating acoustic trauma in a subject, methods of increasing sound evoked feedback inhibition in a subject, and methods of preventing noise-induced and/or age-related hearing loss in a subject. The methods include the intracochlear or other administration of the vectors including a polynucleotide encoding an α9L9′T protein or of the pharmaceutical compositions comprising the same.

### CROSS-REFERENCE TO RELATED APPLICATIONS

This application claims benefit of priority under 35 U.S.C. § 119(e) of U.S. Provisional Application No. 63/410,543, filed Sep. 27, 2022. The disclosure of the prior application is considered part of and is herein incorporated by reference in the disclosure of this application in its entirety.

### STATEMENT REGARDING GOVERNMENT FUNDING

This invention was made with government support under Grant No. DC001508, awarded by the National Institutes of Health. The government has certain rights in the invention.

### BACKGROUND OF THE INVENTION

### Field of the Invention

The present invention relates generally to gene therapy to preserve hearing, and more specifically to the induction of the expression of an α9L9′T protein into the cochlea to protect against hearing loss and acoustic trauma.

### Background Information

Mechanosensory hair cells of vertebrates are subject to cholinergic inhibition by efferent brainstem neurons. Cholinergic olivocochlear efferents are driven by sound to inhibit outer hair cells and provide gain control of the mature cochlea. Efferent feedback is thought to extend dynamic range, reduce noise masking, enhance selective attention, and contribute to perceptual learning. In addition, transient efferent inhibition of early postnatal inner hair cells may contribute to developmental maturation of the auditory pathway.

Beyond these actions, animal studies support a role for olivocochlear inhibition in protection from acoustic trauma. Supportive evidence has been provided by mouse models in which either the α9 or α10 subunits of the hair cell's acetylcholine receptor was knocked out or altered to produce a gain of function nAChR (α9L9′T). Noise-induced and age-related hearing loss are exacerbated in α9 knockout mice but mitigated in the α9L9′T gain of function mice while susceptibility to noise damage correlates with expression of the α9 nAChR subunit. These protective effects of efferent feedback make it a promising target for therapeutic intervention. For example, positive allosteric modulators of the hair cell's nAChR could provide a drug regimen to protect from hearing loss. Another strategy is to employ gene therapy to convert hair cell nAChRs to the gain of function phenotype. The present disclosure describes viral transduction for genetic rescue of ‘efferent knockout’ mice as proof of principle for generalized acoustic prophylaxis.

Gene therapy for sensory loss broke ground with application to retinitis pigmentosa. Efforts to repair the inner ear have focused largely on the rescue of single gene mutations in mouse models, with some examples including vesicular glutamate transporters, the presynaptic release protein otoferlin and mechano-transduction channels. These strategies aim to correct specific, monogenic causes of congenital hearing loss. Progress is beginning for ‘generic’ noise-induced and age-related hearing loss, with presbycusis being by far the most prevalent pathology in industrialized societies. Environmental noise exposure can hasten the onset and increase the severity of presbycusis. Strengthening the naturally occurring sound-evoked feedback inhibition will provide enhanced automatic gain control, matching the level of protection to the level of threat.

### SUMMARY OF THE INVENTION

The present invention is based on the seminal discovery that the induction of the expression of an α9L9′T protein into the cochlea protects against hearing loss such as acoustic trauma-, noise-induced and/or age-related hearing loss.

In one embodiment, the invention provides a method of preventing hearing loss in a subject including administering to the subject a pharmaceutical composition comprising an effective amount of a polynucleotide encoding an α9L9′T protein, thereby protecting against or treating acoustic trauma in the subject.

In one aspect, the hearing loss is selected from the group consisting of age-related hearing loss, noise-induced hearing loss, acoustic trauma induced hearing loss, and a combination thereof. In another aspect, the subject: (i) suffers from progressive hearing loss, (ii) is exposed to noise or acoustic trauma, (iii) is treated for a hearing-loss related pathology, or (iv) any combination thereof.

In another embodiment, the invention provides a method of protecting against or treating acoustic trauma in a subject including administering to the subject a pharmaceutical composition including an effective amount of a polynucleotide encoding an α9L9′T protein, thereby protecting against or treating acoustic trauma in the subject.

In one aspect, protecting against or treating acoustic trauma includes increasing sound evoked feedback inhibition, preventing noise-induced and/or preventing age-related hearing loss. In another aspect, administering includes intracochlear injection. In one aspect, administering includes intratympanic injection. In some aspects, administering includes posterior semicircular canal injection. In other aspects, administering includes round window injection. In one aspect, increasing sound evoked feedback inhibition, preventing noise-induced and/or preventing age-related hearing loss includes mitigating noise-induced pattern of threshold shift. In some aspects, mitigating noise-induced pattern of threshold shift includes reducing temporary or permanent auditory brainstem response (ABR) and distortion product otoacoustic emissions (DPOAEs) thresholds shifts and sensitivity to acoustic trauma. In various aspects, reducing temporary or permanent ABR threshold shifts includes reducing maximal amplitude of ABR wave 1. In many aspects, reducing temporary or permanent ABR threshold shifts includes decreasing ABR wave 1 latency. In other aspects, reducing temporary or permanent ABR threshold shifts includes reducing or eliminating ABR shifts at all frequencies. In one aspect, the method includes limiting hearing loss to mid and upper frequencies. In another aspect, the method further includes administering or using other therapies to protect against or treat acoustic trauma. In some aspects, other therapies include ear covering, hearing aids, single gene repair and/or small molecule therapy. In one aspect, the subject has noise-induced, age-related hearing loss, and/or a pathology exacerbated by noise.

In another embodiment, the invention provides a method of increasing sound evoked feedback inhibition in a subject including administering to the subject a pharmaceutical composition including an effective amount of a polynucleotide encoding an α9L9′T protein, thereby increasing sound evoked feedback inhibition in the subject.

In an additional embodiment, the invention provides a method of preventing noise-induced and/or age-related hearing loss in a subject including administering to the subject a pharmaceutical composition including an effective amount of a polynucleotide encoding an α9L9′T protein, thereby preventing noise-induced and/or age-related hearing loss in the subject.

In one aspect, the polynucleotide encoding an α9L9′T protein is in a vector. In some aspects, the vector is a viral vector. In other aspects, the viral vector is an AAV vector. In various aspects, the AAV vector is an AAV2 vector.

In a further embodiment, the invention provides a vector including a polynucleotide encoding an α9L9′T protein.

In another embodiment, the invention provides a pharmaceutical composition including a vector including a polynucleotide encoding an α9L9′T protein, and a pharmaceutically acceptable carrier.

In one aspect, the pharmaceutically acceptable carrier is a gel or a nanoparticle.

### BRIEF DESCRIPTION OF THE DRAWINGS

FIG. 1 a schematic illustrating the experimental design and timeline.

FIGS. 2A-2B illustrate the viral transduction of green fluorescent protein (GFP) in cochlea. FIG. 2A is a photograph illustrating the middle turn of a P21 cochlea. FIG. 2B is a photograph illustrating an enlargement of boxed region from FIG. 2A.

FIGS. 3A-3F illustrate noise-induced hearing loss in α9 null mice. FIG. 3A is a graph illustrating ABR threshold prior to acoustic trauma. FIG. 3B is a graph illustrating one day post acoustic trauma thresholds among all three cohorts of mice. FIG. 3C is a graph illustrating fourteen days after trauma ABR thresholds. FIG. 3D is a graph illustrating mean (±SEM) ABR threshold for 24 uninjected α9-null C57Bl/6J mice, before (circles), one day post (squares), and 14 days post (triangles) acoustic trauma. FIG. 3E is a graph illustrating mean (±SEM) ABR threshold for ABR threshold for 10 α9-null C57Bl/6J mice injected with GFP virus only before (circles), one day post (squares), and 14 days post (triangles) acoustic trauma. FIG. 3F is a graph illustrating mean (SEM) ABR threshold for 31 α9-null C57Bl/6J mice injected with α9L9′T virus plus GFP before (circles), one day post (squares), and 14 days post (triangles) acoustic trauma. Statistical significance for all panels from multiple unpaired t-tests with Welch correction, *=p<0.05, **=p<0.01, ***=p<0.001.

FIG. 4 is a graph illustrating the impact of acoustic trauma on distortion product oto-acoustic emissions (DPOAEs) in 3 mice injected with a combination of α9L9′T-containing and GFP-containing viruses before (circles), one day post (squares), and 14 days post (triangles) acoustic trauma.

FIGS. 5A-5F illustrate ABR wave 1 amplitudes for saturating loud tones. FIG. 5A is a graph illustrating baseline wave 1 amplitudes for all experimental cohorts prior to acoustic trauma average (±SEM). FIG. 5B is a graph illustrating wave 1 amplitude 14 days post trauma for all cohorts. FIG. 5C is a graph illustrating the ratio of wave 1 amplitude at day 14 over the pre-trauma value for all cohorts. FIG. 5D is a graph illustrating the average (±SEM) of wave 1 amplitude in 24 uninjected mice before (circles), one day post (squares), and 14 days post (triangles) acoustic trauma. FIG. 5E is a graph illustrating wave 1 amplitude of 10 GFP injected mice before (circles), one day post (squares), and 14 days post (triangles) acoustic trauma. FIG. 5F is a graph illustrating wave 1 amplitude of 30 α9L9′T injected mice before (circles), one day post (squares), and 14 days post (triangles) acoustic trauma. Multiple unpaired t-tests with Welch correction for all panels, *=p<0.05, **=p<0.01, ***p<0.001.

FIGS. 6A-6F illustrate ABR Wave 1 Latencies. FIG. 6A is a graph illustrating the average (±SEM) for ABR wave 1 latency prior to acoustic trauma in uninjected α9 null, GFP virus only injected and α9L9′T-containing virus plus GFP-containing virus injected. FIG. 6B is a graph illustrating ABR wave 1 latencies 14 days post trauma. FIG. 6C is a graph illustrating ABR wave 1 latency for uninjected α9 null mice, before, one- and 14-days post trauma. FIG. 6D is a graph illustrating ABR wave 1 latency for GFP injected α9 null mice, before, one- and 14-days post trauma. FIG. 6E is a graph illustrating ABR wave 1 latency for α9L9′T injected α9 null mice, before, one- and 14-days post trauma. FIG. 6F is a graph illustrating the average (±SEM) latency shift at 1 day post trauma in all cohorts. Multiple t-tests with Welch correction, *p<0.05, **p<0.01, ***p<0.001 for all panels.

FIGS. 7A-7F illustrate afferent synapses of inner hair cells (mean±SEM). FIG. 7A is a graph illustrating mean number of inner hair cells per condition. FIG. 7B is a graph illustrating synapse counts prior to acoustic trauma for all cohorts. FIG. 7C is a graph illustrating two weeks post acoustic trauma inner hair cells from cochleas for all cohorts. FIG. 7D is a graph illustrating the number of afferent synapses two weeks post trauma in inner hair cells of uninjected mice. FIG. 7E is a graph illustrating the number of afferent synapses two weeks post trauma in inner hair cells of GFP injected mice. FIG. 7F is a graph illustrating the number of afferent synapses two weeks post trauma in inner hair cells of α9L9′T injected mice.

FIGS. 8A-8F illustrate ribbons in outer hair cells. FIG. 8A is a graph illustrating the average number of OHCs in each experimental cohort. FIG. 8B is a graph illustrating ribbon counts in outer hair cells at five frequency loci in cochleas before acoustic trauma. FIG. 8C is a graph illustrating ribbons count in outer hair cells at five frequency loci in cochleas 14 days post trauma. FIG. 8D is a graph illustrating ribbon counts in outer hair cells of uninjected cochleas. FIG. 8E is a graph illustrating ribbon counts in outer hair cells of GFP injected cochleas. FIG. 8F is a graph illustrating ribbon counts in outer hair cells of α9L9′T injected cochleas.

FIGS. 9A-9F illustrate efferent synapses on outer hair cells. FIG. 9A is a graph illustrating the average number of OHCs in each experimental cohort. FIG. 9B is a graph illustrating efferent synaptic volumes per OHC without acoustic trauma. FIG. 9C is a graph illustrating efferent synaptic volumes 14 days after acoustic trauma. FIG. 9D is a graph illustrating synaptic volumes in uninjected cochleas with and without noise exposure. FIG. 9E is a graph illustrating synaptic volumes in GFP only injected cochleas with and without noise exposure. FIG. 9F is a graph illustrating synaptic volumes in α9L9′T plus GFP injected cochleas with and without noise exposure. Multiple t-tests with Welch correction, *=p<0.05, **=p<0.01, ***=p<0.001.

FIG. 10 is a graph illustrating the threshold for the auditory brainstem response ABR in WT mice uninjected or injected with HA α9L9′T.

### DETAILED DESCRIPTION OF THE INVENTION

The present invention is based on the seminal discovery that the induction of the expression of an α9L9′T protein into the cochlea protects against hearing loss such as acoustic trauma-, noise-induced and/or age-related hearing loss.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a,”“ ” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the invention provides a method of preventing hearing loss in a subject including administering to the subject a pharmaceutical composition including an effective amount of a polynucleotide encoding an α9L9′T protein, thereby preventing hearing loss in the subject.

Nicotinic acetylcholine receptors, or nAChRs, are receptor polypeptides that respond to the neurotransmitter acetylcholine. Nicotinic receptors also respond to drugs such as the agonist nicotine. They are found in the central and peripheral nervous system, muscle, and many other tissues of many organisms. At the neuromuscular junction they are the primary receptor in muscle for motor nerve-muscle communication that controls muscle contraction. In the peripheral nervous system: (1) they transmit outgoing signals from the presynaptic to the postsynaptic cells within the sympathetic and parasympathetic nervous system, and (2) they are the receptors found on skeletal muscle that receive acetylcholine released to signal for muscular contraction. The nicotinic receptors are considered cholinergic receptors since they respond to acetylcholine. Nicotinic receptors get their name from nicotine which does not stimulate the muscarinic acetylcholine receptors but selectively binds to the nicotinic receptors instead. Nicotinic receptors, with a molecular mass of 290 kDa, are made up of five subunits, arranged symmetrically around a central pore. Each subunit comprises four transmembrane domains with both the N- and C-terminus located extracellularly. They possess similarities with GABAA receptors, glycine receptors, and the type 3 serotonin receptors (which are all ionotropic receptors), or the signature Cys-loop proteins.

In vertebrates, nicotinic receptors are broadly classified into two subtypes based on their primary sites of expression: muscle-type nicotinic receptors and neuronal-type nicotinic receptors. In the muscle-type receptors, found at the neuromuscular junction, receptors are either the embryonic form, composed of α1, β1, γ, and δ subunits in a 2:1:1:1 ratio ((α1)2β1γδ), or the adult form composed of α1, β1, δ, and ε subunits in a 2:1:1:1 ratio ((α1)2β1δε). The neuronal subtypes are various homomeric (all one type of subunit) or heteromeric (at least one α and one β) combinations of twelve different nicotinic receptor subunits: α2-α10 and β2-β4. Examples of the neuronal subtypes include: (α4)3(β2)2, (α4)2(β2)3, (α3)2(β4)3, α4α6β3(β2)2, (α7)5, and many others. The nAChR subunits have been divided into 4 subfamilies (I-IV) based on similarities in protein sequence. In addition, subfamily III has been further divided into 3 types. Neuronal nAChRs are transmembrane proteins that form pentameric structures assembled from a family of subunits composed of α₂-α₁₀ and β₂-β₄. When expressed alone, α₇, α₈, α₉, and α₁₀ are able to form functional receptors, but other a subunits require the presence of β subunits to form functional receptors.

Neuronal acetylcholine receptor subunit alpha-9 (α9), also known as nAChRα9, is a protein that in humans is encoded by the CHRNA9 gene. The protein encoded by this gene is a subunit of certain nicotinic acetylcholine receptors (nAChR). α9 subunit-containing receptors are notably blocked by nicotine. The role of this antagonism in the effects of tobacco are unknown. This gene is a member of the ligand-gated ionic channel superfamily and nicotinic acetylcholine receptor gene family. It encodes a plasma membrane protein that forms homo- or hetero-oligomeric divalent cation channels. This protein is involved in cochlear hair cell function and is expressed in both the inner (IHCs) and outer hair cells (OHCs) of the adult cochlea, although expression levels in adult inner hair cells is low. The activation of the alpha9/10 nAChR is via olivocochlear activity, represented by cholinergic efferent synaptic terminals originating from the superior olive region of the brainstem. The protein is additionally expressed in keratinocytes, the pituitary gland, B-cells, and T-cells.

Cholinergic, medial olivocochlear brainstem neurons innervate the adult cochlea to inhibit mechanosensory outer hair cells. These efferent neurons are themselves driven by sound and so constitute a negative feedback loop to regulate cochlear sensitivity. Efferent cholinergic feedback also provides protection against acoustic trauma. That protection is absent from nAChR null mice (lacking the ligand-binding α9 subunit) and is stronger in knock-in mice expressing a gain-of-function point mutation (α9L9′T) of the hair cell's nAChR.

The methods described herein include administering a pharmaceutical composition including an effective amount of a polynucleotide encoding an α9L9′T protein to prevent hearing loss.

As used herein, the term “α9L9′T protein” refers to the α9 subunit of the acetylcholine receptor (AChR) containing a gain-of-function point mutation (α9L9′T) of the hair cell's nAChR.

Acoustic trauma is an injury to the inner ear that is often caused by exposure to a high-decibel noise. It can occur after exposure to a single, very loud noise or from exposure to noises at significant decibels over a longer period of time. Some injuries to the head can cause acoustic trauma if the eardrum is ruptured or if other injuries to the inner ear occur. Acoustic trauma damages the way that these vibrations transmitted from the eardrum to the brain are handled, resulting in hearing loss. Loud sound moving into the inner ear can cause a threshold shift, which can trigger hearing loss. Three important factors have a role in acoustic trauma: the intensity of sound measured in decibels, the pitch or frequency of the sound (higher frequencies are more damaging), and the total time the person was exposed to the sound.

Progressive hearing loss is common and can affect anyone in the general population. Hearing loss can be temporary (e.g., the loss can be recovered, and hearing can be considered restored, usually quickly after exposure) or permanent (e.g., the loss cannot be recovered, and hearing cannot be restored). External factors such as age or acoustic trauma exposure can exacerbate or accelerate hearing loss. In the context of the present invention, the therapeutic methods described herein are generally intended to be administered before exposure to a factor that can induce initial hearing loss, or further hearing loss (when some hearing loss has already occurred). It is generally understood in the art that permanent hearing loss cannot be recovered (nor cured), however, the present invention provides for therapeutic tools to prevent future hearing loss, either age-related hearing loss (or presbycusis) or acoustic trauma/noise induced hearing loss.

By “preventing” hearing loss in a subject, it is meant that the subject is susceptible to hearing loss, and that the therapeutic pharmaceutical composition of the invention is to be administered to the subject before exposure to a factor that is likely to induce hearing loss. As detailed below in the examples, the pharmaceutical composition of the invention has been demonstrated to be efficient at protecting against a hearing loss that would otherwise occur in the absence of the administration of the pharmaceutical composition. Therefore, by “preventing” it is meant that the pharmaceutical composition of the invention protects against future hearing loss. As used herein, preventing is also meant to include the reduction of the hearing loss that would otherwise occur. In various aspects, the methods described herein reduce hearing loss by at least about 1%. For example, the methods described herein reduce hearing loss by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more.

As used herein, the hearing loss can be induced or result from exposure of the subject to one or more external factors. “External factors” as used herein are meant to include age, noise, (such as chronic or recurrent noise exposure), acoustic trauma (such as chronic or recurrent exposure to extreme/very loud noise), or any combination thereof.

In one aspect, the hearing loss is selected from the group consisting of age-related hearing loss, noise-induced hearing loss, acoustic trauma induced hearing loss, and a combination thereof.

Progressive hearing loss is common and can affect anyone in the general population. Therefore, virtually anyone could benefit from the methods described herein, at least for prophylactic purposes. Some subjects are however more likely than others to develop progressive hearing loss and are therefore easier to identify as subjects that could benefit from the methods described herein. notably, the methods described herein are particularly useful for subjects that (i) have already been identified as suffering from progressive hearing loss, (ii) are currently, have been or will be exposure to chronic or recurrent noise or acoustic trauma, (iii) are currently, have been or will be treated for a hearing loss related pathology, or (iv) fall into more than one or the above-described situations.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters, and guinea pigs), cats, dogs, rabbits, farm animals including cows, horses, goats, sheep, pigs, chickens, etc., and primates (including monkeys, chimpanzees, orangutans, and gorillas) are included within the definition of subject. In one aspect, the subject has noise-induced, age-related hearing loss, and/or a pathology exacerbated by noise.

As used herein, subject suffering from progressive hearing loss include subject for whom a hearing loss has been formally identified and diagnosed, and who are therefore known to be at risk of losing further hearing loss over time, at a pace that would be variable based on the cause for the hearing loss, and the exposure to accelerating/exacerbating factors. For example, subjects suffering from progressive hearing loss include elderly subjects suffering from age-related progressive hearing loss, subjects suffering from inherited pathology that directly or indirectly induces progressive hearing loss, and subjects suffering from a non-genetic or inherited pathology that directly or indirectly induces progressive hearing loss.

In one aspect, the subject (i) suffers from progressive hearing loss, (ii) is exposed to noise or acoustic trauma, (iii) is treated for a hearing-loss related pathology, or (iv) any combination thereof.

The term “treatment” is used interchangeably herein with the term “therapeutic method” and refers to both 1) therapeutic treatments or measures that slow down, lessen symptoms of, and/or halt progression of a diagnosed pathologic conditions or disorder, and 2) and prophylactic/preventative measures, which protect against the occurrence of the pathologic condition. Those in need of treatment may include individuals already having a particular medical disorder (e.g., hearing loss) as well as those who may ultimately acquire the disorder (i.e., those needing preventive measures for hearing loss).

The terms “therapeutically effective amount,” “effective dose,” “therapeutically effective dose,” “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment or prevention of acoustic trauma) . The effective amount can be determined as described herein.

The pharmaceutical composition used in the methods described herein is meant to be administered to the subject. The terms “administration of” and or “administering” should be understood to mean providing a pharmaceutical composition in a therapeutically effective amount to the subject in need of treatment. Administration routes can be enteral, topical, or parenteral. As such, administration routes include but are not limited to intratympanic, intracochlear, intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

The pharmaceutical composition describes herein is meant to be administered as a single therapy, or in combination with any complementary therapy used by the subject to protect or treat their hearing loss.

In some aspects administration can be in combination with one or more additional therapeutic agents. The phrases “combination therapy,” “combined with” and the like refer to the use of more than one medication or treatment simultaneously to increase the response. The composition of the present invention might for example be used in combination with other drugs, treatment or protecting assisting material in use for the management of hearing loss.

For example, subjects suffering from progressive hearing loss can be treated or plan to be treated for their hearing loss with any of the therapeutic tools available for each case, including using pharmaceutical treatments, protective material, hearing aids, and any combination thereof. For example, subjects suffering from a monogenic disease responsible for progressive hearing loss can be treated with gene therapy to replace the defective protein in the ear; subjects suffering from age-related hearing loss can use hearing aids, and subjects recurrently exposed to noise or acoustic trauma can use hearing protection equipment.

Specifically, the administration of the pharmaceutical composition of the invention to subject can be in combination with any of those therapies or tools. Such therapies or tools can be administered prior to, simultaneously with, or following administration of the composition of the present invention.

That is, because hearing loss is a progressive disease, the use of protective equipment, the use of hearing aids, or the treatment with gene therapy for example does not necessarily prevent from any further hearing loss, which in those affected subjects is usually accelerated or exacerbated by exposure to risk factors such as aging, noise and/or acoustic trauma. The methods described herein can therefore be used as auxiliary or complementary to prevent any additional/future hearing loss.

In another embodiment, the invention provides a method of protecting against or treating acoustic trauma in a subject including administering to the subject a pharmaceutical composition including an effective amount of a polynucleotide encoding an α9L9′T protein, thereby protecting against or treating acoustic trauma in the subject.

By “protecting against” or “treating” acoustic trauma in a subject, it is meant that the methods described herein are useful at preventing hearing loss resulting from acoustic trauma and providing a solution to restore at least partially hearing loss resulting from temporary acoustic trauma, respectively. “Treating” as used herein is meant to be understood as “preventing” hearing loss is a subject, “protecting” against future hearing loss, or “reducing” hearing loss. In various aspects, the methods described herein reduce hearing loss by at least about 1%. For example, the methods described herein reduce hearing loss by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more.

The terms “administration of” and or “administering” should be understood to mean providing a pharmaceutical composition in a therapeutically effective amount to the subject in need of treatment. Administration routes can be enteral, topical, or parenteral. As such, administration routes include but are not limited to intratympanic, intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In one aspect, administering includes intracochlear injection. In another aspect, administering includes intratympanic injection. In various aspects, administering includes posterior semicircular canal injection. In other aspects, administering includes round window injection.

In one aspect, protecting against or treating acoustic trauma includes increasing sound evoked feedback inhibition, preventing noise-induced and/or preventing age-related hearing loss.

In one aspect, increasing sound evoked feedback inhibition, preventing noise-induced and/or preventing age-related hearing loss includes mitigating the noise-induced pattern of threshold shift.

In some aspects, mitigating the noise-induced pattern of threshold shift includes reducing temporary or permanent auditory brainstem response (ABR) and distortion product otoacoustic emissions (DPOAEs) threshold shifts and sensitivity to acoustic trauma.

The auditory brainstem response (ABR) test tells how the inner ear (i.e., the cochlea), and the brain pathways for hearing are working. It may also be referred to as an auditory evoked potential (AEP). The test is used with children or others who cannot complete a typical hearing screening. The ABR is also used if your symptoms might be due to hearing loss in the brain or in a brain pathway. During a test, electrodes are put on a subject's head and connected to a computer. They record brain wave activity in response to sounds heard through earphones.

Distortion product otoacoustic emissions (DPOAEs) reflect outer hair cell integrity and cochlear function. When used in the audiology clinic, they are an effective diagnostic tool and can detect hearing loss with accuracy. DPOAEs are easily and rapidly recorded in newborns and children and provide basic hearing screening information as well as detailed diagnostic information in cases of suspected hearing loss. DPOAEs also provide hearing scientists with a frequency-specific and noninvasive probe of the cochlea and cochlear amplifier function. Sophisticated and complex DPOAE-based experimental paradigms have been developed and applied to address scientific questions about cochlear function in humans.

Outer hair cells (OHCs) that are lined up in three rows atop the basilar membrane begin to elongate and contract at rates that are well beyond contraction rates for muscle tissue. Their motility is triggered by a significant change in resting potential. This voltage change is produced by the traveling wave motion; therefore, only those OHCs that are maximally stimulated (on the upswing of the traveling wave) become motile. And, because they are motile in only a focal region of the membrane, the displacement around this narrow cochlear region (i.e., characteristic frequency) is made larger, powered by OHCs that are pulling downward on the tectorial membrane and upward on the basilar membrane. This mechanical amplification of basilar membrane motion sends a clear and robust message to the brain about the acoustic input. This robust vibration pattern on the basilar membrane translates into excellent detection of sound. Enhanced membrane displacement in a focal region, around the tip of the traveling wave and just basal to it, also produces a more highly tuned signal, which translates into excellent frequency resolution.

This OHC-mediated amplification process produces an enhanced representation of the auditory signal to the brain and has been collectively termed the “cochlear amplifier” (Davis, 1983). The cochlear amplifier (CA) works to improve perception of sounds that are low-to-moderate in level only. The cochlear amplifier's effectiveness saturates with high level input. It is not difficult to hypothesize the purpose of this amplifier and why it might have evolved. Without sensitive detection and exquisite frequency resolution, humans would have trouble detecting some of the critical nuances of speech; nuances that are required for adequate speech perception and discrimination. In fact, when an individual has mild-to-moderate amounts of sensorineural hearing loss he or she has typically lost OHCs (cochlear amplifier) and often scores poorly on speech discrimination tasks in noise.

In the process of amplifying and enhancing sound for efficient decoding by the brain, the cochlea creates a byproduct. Just like sound that comes into the ear from an external source, the cochlear byproduct produces its own physical vibration along the basilar membrane. The DPOAE is generated by the cochlea when the ear is presented with two simultaneous pure tones (f1 and f2). The DPOAE travels from its generation site on the basilar membrane around the f1 and f2 frequency to the region of its own characteristic frequency. It also travels in reverse direction from its generation site, through the middle ear, and into the ear canal. The cochlear-generated distortion becomes an acoustic product once it is in the ear canal and can be measured with a sensitive microphone placed at the auditory meatus. When the DPOAE is present in the ear canal, it indicates that the mechanism generating it (i.e., the cochlear amplifier) is functional; when the DPOAE is absent, it indicates that the amplifier is nonfunctional or dysfunctional and that there is hearing loss. In this way, a distortion tone produced by the ear as a byproduct and measured in the ear canal has the useful characteristic of reflecting cochlear integrity.

In various aspects, reducing temporary or permanent ABR threshold shifts includes reducing maximal amplitude of ABR wave 1. In many aspects, reducing temporary or permanent ABR threshold shifts includes decreasing ABR wave 1 latency. In other aspects, reducing temporary or permanent ABR threshold shifts includes reducing or eliminating ABR shifts at all frequencies. In one aspect, the method includes limiting hearing loss to mid and upper frequencies. By reducing maximal amplitude of ABR wave 1 it is also meant that that maximal amplitude of ABR wave 1 is preserved (e.g., no increase is observed either, even though a decrease is not necessarily observed).

In another aspect, the method further includes administering or using other therapies to protect against or treat acoustic trauma.

In some aspects administration can be in combination with one or more additional therapeutic agents. The phrases “combination therapy,” “combined with” and the like refer to the use of more than one medication or treatment simultaneously to increase the response. The pharmaceutical composition of the present invention might for example be used in combination with other drugs or treatment in use to treat or prevent acoustic trauma. Specifically, other therapies used to treat or prevent acoustic trauma in a subject can be in combination with the pharmaceutical composition of the present invention. Such therapies can be administered prior to, simultaneously with, or following administration of the composition of the present invention.

In some aspects, other therapies include ear covering, hearing aids, single gene repair and/or small molecule therapy.

Gene repair, or molecular therapies include gene replacement, gene augmentation, gene editing, post-translational modifications, and alternative splicing. The transfer of genetic material into cells can be achieved via viral or non-viral vectors, including adenovirus, adeno-associated virus (AAV), herpes simplex virus I, vaccinia virus and non-viral technologies such as lipid gene transfer systems. Several gene candidates are investigated for their impact on the treatment of hearing loss. They include but are not limited to the Whirlin gene (required for actin polymerization in hair cell stereocilia), TMC1 (a gene required for hair cell transduction channel function), vesicular glutamate transporter type 3 (VGLUT3, required for synaptic transmission in hair cells), and Atoh1 (a gene encoding a basic helix-loop-helix transcription factor).

In another embodiment, the invention provides a method of increasing sound evoked feedback inhibition in a subject including administering to the subject a pharmaceutical composition including an effective amount of a polynucleotide encoding an α9L9′T protein, thereby increasing sound evoked feedback inhibition in the subject.

In an additional embodiment, the invention provides a method of preventing noise-induced and/or age-related hearing loss in a subject including administering to the subject a pharmaceutical composition including an effective amount of a polynucleotide encoding an α9L9′T protein, thereby preventing noise-induced and/or age-related hearing loss in the subject.

In one aspect, the polynucleotide encoding an α9L9′T protein is in a vector.

As used herein, the term “nucleic acid” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic acid can be employed for introduction into, i.e., transfection of, cells, in particular, in the form of RNA which can be prepared by in vitro transcription from a DNA template. The RNA can moreover be modified before application by stabilizing sequences, capping, and polyadenylation.

The terms “peptide,” “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The term “vector,” “expression vector,” or “plasmid DNA” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences can be operatively linked, such as a gene encoding a protein of interest, one or more protein tags, functional domains, and the like.

Vectors suitable for use in preparation of proteins and/or protein conjugates include those selected from baculovirus, phage, plasmid, phagemid, cosmid, fosmid, bacterial artificial chromosome, viral DNA, Pl-based artificial chromosome, yeast plasmid, and yeast artificial chromosome. Polynucleotides can be delivered to cells (e.g., a plurality of different cells or cell types including target cells or cell types and/or non-target cell types) in a vector (e.g., an expression vector). Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viral vectors have several advantages for delivery of nucleic acids, including high infectivity and/or tropism for certain target cells or tissues. In some cases, a viral vector can be used to deliver a polynucleotide described herein.

Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest. In the expression vectors, regulatory elements controlling transcription can be generally derived from mammalian, microbial, viral or insect genes. The ability to replicate in a host, usually conferred by an origin of replication, and a selection gene to facilitate recognition of transformants may additionally be incorporated. Those of skill in the art can select a suitable regulatory region to be included in such a vector.

The term “AAV” is an abbreviation for adeno-associated virus and can be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV12, AAVDJ, rhlO, derivatives and hybrids thereof, avian AAV, bovine AAV, canine AAV, equine AAV, primate AAV, non-primate AAV, and ovine AAV. Additionally, any engineered or variant derived from ancestral AAV sequence reconstruction can be used as a vector. The genomic sequences of various serotypes of AAV, as well as the sequences of the native terminal repeats (TRs), Rep proteins, and capsid subunits are known in the art. Such sequences may be found in the literature or in public databases such as GenBank. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). The term rAAV vector encompasses both rAAV vector particles and rAAV vector plasmids. An rAAV vector may either be single stranded (ssAAV) or self-complementary (scAAV). An “AAV virus” or “AAV viral particle” or “rAAV vector particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector.” Thus, production of rAAV particle necessarily includes production of rAAV vector, as such a vector is contained within an rAAV particle. In various aspects, the AAV vector is an AAV2 vector.

In a further embodiment, the invention provides a vector including a polynucleotide encoding an α9L9′T protein.

In another embodiment, the invention provides a pharmaceutical composition including a vector including a polynucleotide encoding an α9L9′T protein, and a pharmaceutically acceptable carrier.

The vector can be in a pharmaceutical composition, along with a pharmaceutically acceptable carrier. By “pharmaceutically acceptable” it is meant that the carrier, diluent, or excipient must be compatible with the other ingredients of the formulation and not deleterious to the recipient thereof. Pharmaceutically acceptable carriers, excipients or stabilizers are well known in the art, for example Remington's Pharmaceutical Sciences, 16^{th} edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil, and dimethyl sulfoxide (DMSO). In one aspect, the pharmaceutically acceptable carrier is a gel or a nanoparticle.

Presented below are examples discussing α9L9′T gene therapy to prevent and treat hearing loss contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Material and Methods

### α9L9′T AAV2.7m8

The α9L9′T sequence was inserted into AAV2.7m8 (obtained via MTA from Addgene. This is a modified form of the AAV2 virus containing a 10-amino acid peptide inserted at position 588 of the AAV2 capsid protein sequence (Dalkara, Byrne et al. 2013). AAV2.7m8 was shown to provide efficient transduction of inner and outer cochlear hair cells with green fluorescent protein-GFP (Isgrig, McDougald et al. 2019). Control experiments were conducted with that same GFP-containing AAV2.7m8. Mouse α9L9′T cDNA was incorporated into a plasmid and incorporated into the AAV2.7m8 backbone. The resulting viral vector, (AAV2.7m8.CAG.nAChRα9L9′T.bGH) was provided for use at 3.26×10¹³ viral copies/ml. This was subdivided into 100 μl aliquots and stored at −80° C. till used. Each aliquot was stored at 4° C. (i.e., not refrozen) till exhausted.

Mice (α9 nulls and wildtype). A9-null (homozygous knockout) mice have qualitatively normal efferent innervation, but no cochlear inhibition. In the altered gene the ligand-binding site, the first and second transmembrane domains, and a portion of the third transmembrane domain of the α9 coding region were excised. The original mice (129S6 background) were subsequently backcrossed onto C57BL/6J for more than ten generations and breeding colonies maintained in the Johns Hopkins Research Animal Resource. Mice C57BL/6J (RRID: IMSR_JAX:000664) mice purchased from Jackson Laboratories, and α9 knockouts (C57 background) were bred and maintained in the Johns Hopkins University School of Medicine Research Animal Resource facility. Mice were placed on a 12-hour light-dark cycle, fed an autoclaved Teklad diet, and housed in cages with automatic water and filtered air. All experiments were carried out under protocols approved by the Institutional Animal Care and Use Committee. Male and female mice were used in all experiments to avoid sex-specific variation.

### Injection Protocol

1.1 μl of virus (at 10¹³ viral particles/ml) was injected into the posterior semi-circular canal. The α9L9′T-containing virus was always injected together with the GFP-containing virus (i.e., two independent viruses). To control for the impact of surgery and virus injection per se, a cohort of mice were injected with GFP-containing virus only. Mouse pups (0-2 days old, P0-P2) were anaesthetized by cooling. Under a stereo dissecting scope, a small dorsal-ventral post-auricular incision was made through skin and muscle to expose the posterior semi-circular canal as a darker tube surrounded by whiter tissue. The injection pipette was used to penetrate through the soft bone in young animals. Repeated, brief pressure pulses were used to inject the viral aliquot over proximately 40 seconds. The pipette was removed, the skin sutured together over the incision and the animals left to recover on a warming pad. The wound site was disinfected with betadine and the entire procedure carried out under sterile conditions.

### Fluorophore Conjugated Conotoxin Peptide (Cy3-RgIA-5727) Labeling of Cochlear nAChRs

The fluorophore conjugated modified conopeptide Cy3-RgIA-5727 labels hair cell nAChRs in unfixed tissue. Thus, live cochlear tissue (divided into apical, middle, and basal turns) from control and virus-injected mice was dissected from the isolated otic capsule and secured with a thin spring clip fashioned from minuten nadeln cemented to a glass coverslip. The live tissue was exposed to 10-250 nM Cy3-RgIA-5727 in artificial perilymph ((mM): 5.8 KCl, 144 NaCl, 1.3 CaCl2, 0.9 MgCl2, 0.7 NaH2PO4, 5.6 D-glucose, and 10 HEPES (300 mOsm, pH 7.4, adjusted with NaOH) for 15 minutes. The vital dye FM4-64 (10-50 μM) was used to label and visualize hair cells. The labeled tissue was examined on a Nikon A1R-MP upright confocal microscope and z-stack image series acquired using Nikon Nis Element software. 3-D videos were made with the Movie Maker tool in Nis Element software. Image analysis was conducted by a blinded observer in ImageJ/FIJI, Imaris, or using a virtual reality viewing system (Syglass, Istovisio, Inc.). A second blinded observer recounted the images for comparison.

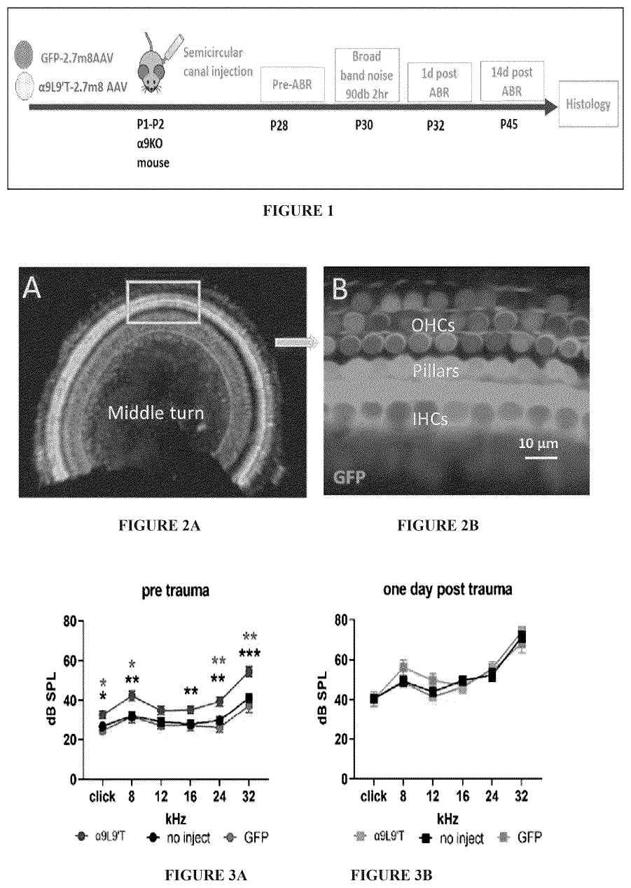

As shown in FIG. 1, α9-null mice were injected with viruses carrying GFP or α9L9′T in combination, GFP virus alone, or not injected. For noise-induced hearing loss studies the uninjected and injected mice were exposed to acoustic trauma four weeks after injection, with ABRs collected before, one day and 14 days after trauma.

### Acoustic Trauma Protocol

C57BL/6J mice were transferred to a low noise satellite housing facility from the day before noise exposure through the week after noise exposure until the endpoint for histology. Due to the susceptibility of C57BL/6J mice to age-related hearing loss, the effect of acoustic trauma was examined before mice were seven weeks old. Awake, unrestrained mice were exposed to 90 dB SPL white noise band (2-20 kHz) for 2 hours to produce a temporary threshold shift of 20-30 dB. Mice were exposed to noise in a reverberant sound attenuating chamber (58 cm×40 cm×30 cm; width, depth, height) with three overhead, Promaster TW47 1200 W dome tweeter speakers that produced maximum energy in the sound spectrum from 2-16 kHz. Speakers were approximately 25 centimeters above the heads of the mice. Broadband noise was generated by two JKT tone and noise generators (KV2 audio, Czech Republic) powered by Neewer nw-100 phantom power sources. The noise generators were connected to two Crown Drivecore XLS2502 amplifiers: one driving the two peripheral speakers in Y input mode, the other driving a central speaker in bridge mode. The sound spectra and decibel level were tested in each set-up using a Larson-Davis LXT sound level meter with a ½-inch free field microphone. Care was taken to measure the sound level at the position of the head of the experimental animals. When not being studied, mice were housed in a ‘quiet’ room where average noise levels (third octave band levels) were below 40 dB SPL.

### Auditory Brainstem Response (ABR) Measurements

The ABR system (Tucker-Davis Technologies), procedures and quantification software used for this study have been previously described (Lauer and May 2011, Lina, and Lauer 2013). Mice were anesthetized with an intraperitoneal injection of 0.1 cc per 20 g body weight of a mixture of ketamine (100 mg/kg) and xylazine (1 mg/kg in PBS). The animals were placed on a gauze covered heating pad in sound attenuating chamber lined with Sonex acoustic foam panels and their eyes were swabbed with petrolatum-based ophthalmic ointment to prevent corneal ulcers. Subdermal platinum electrodes were placed at the level of the vertex of the skull (non-inverting), behind the ventral edge of the left pinna (inverting), and at the base of the tail (ground). Clicks or pure-tone stimuli (512 repetitions 10 to 90 dB in 10 dB steps, 21 stimuli/sec) were used to generate averaged ABR waveforms. The duration of the tonal stimulus was 5 ms, with a 2-ms rise and fall time. A TDT MF1 free field speaker was used to present the stimuli 10 cm from the mouse pinna. The ABR threshold was defined with custom MatLab software (B.J. May) by calculating the averaged peak-to-peak voltage during a 5-ms interval, beginning 1 ms after the onset of the stimulus, compared to the averaged peak-to-peak voltage in a 5-ms window 20 ms after the stimulus (reflecting the baseline physiological noise level). The threshold was determined by interpolating the stimulus level where the peak-to-peak response was greater than 2 standard deviations above the baseline noise. Each threshold determination was confirmed by visual inspection of averaged ABR waveforms.

### Synaptic Immunolabeling

Two weeks after acoustic trauma (P45-46) mice were euthanized by isoflurane anesthesia and decapitation. Otic capsules were removed from the temporal bone and perfused with 4% paraformaldehyde (PFA) in phosphate buffered saline (PBS) through the oval and round windows. All samples were post-fixed in fresh fixative solution for 30 minutes at room temperature (RT), rinsed thoroughly with PBS and the cochlear epithelium micro-dissected. After removal of Reissner's and tectorial membranes, the tissue was permeabilized in PBS containing 0.5% Triton x-100 and 10% normal donkey serum and for 1 to 2 hours at RT. A mixture of primary antibodies was applied to the tissue samples then incubated overnight at RT or at 4° C. Following several rinses in PBS, tissue samples were transferred to AlexaFluor conjugated secondary antibodies (Life technologies, 1:1000 dilution) solution supplemented with the nuclear dye DAPI (1:5000) then incubated for 1 to 2 hours at RT. The samples were rinsed several times in PBS and mounted in FluorSave antifade mounting medium (CalBiochem, San Diego, CA) using grade 1 coverslips recommended for confocal microscopy. Primary antibodies used in this study include Mouse IgG1 anti-CtBP2, (Clone 16, BD Biosciences, AB_39943), rabbit ant-GluAR2/3, (Millipore, AB1506), rabbit anti-synaptophysin (Chemicon #AB9272.). Images were acquired on a LSM700 confocal microscope (Zeiss Axio Imager Z2) using 40× N.A. 1.3 and 63× N.A. 1.4. oil immersion objectives. Images were analyzed using ImageJ/Fiji (RRID: SCR_002285) or Imaris (RRID:SCR_007370).

### Example 2

### Imaging Hair Cell nAChRs with Cy3-RgIA-5727 (α-Conotoxin)

The efficacy of posterior semi-circular canal injection with AAV2.7m8 was determined initially by cochlear expression of green fluorescent protein (GFP) carried by the virus. AAV2.7m8.CAG.mChrna9.IRES.eGFP. bGH was injected into the posterior semi-circular canal at P0-P2. Mice were examined 2-3 weeks later for GFP expression. As illustrated in FIG. 2 injection of virus at postnatal day 0 to 2 (P0-P2), produced widespread expression of GFP throughout the cochlea at P19-21. GFP expression was seen in inner and outer hair cells and in supporting cells that surround inner hair cells.

Viral transduction of α9L9′T into α9-null mice was assessed by labeling hair cell nAChRs with Cy3-conjugated RgIA-5727. RgIA is a modified form of an a-peptide isolated from cone snail venom that binds to and blocks α9-containing nAChRs with high affinity. RgIA conjugated to the fluorophore Cy3 (Cy3-RgIA-5727) was shown to bind irreversibly at hair cell postsynaptic densities opposite presynaptic efferent terminals. Cy3-RgIA-5727 labeled outer hair cells of α9-null mice after viral transduction in a pattern consistent with the known efferent innervation of cochlear tissue. When hair cells were visualized by loading with the permeant fluorescent dye FM4-64, fluorescent Cy3 puncta were observed at the base of outer hair cells in 3-week-old mouse cochleas. Fluorescent puncta also were seen near the cuticular plate where efferent synaptic contacts have been described in electron micrographs and demonstrated by HA-tagged nAChR expression. In addition to labeled synaptic puncta, stereocilia were illuminated in the Cy3 images due to overlapping spectra with FM4-64 (Fisher, Zhang et al. 2021). Following virus injection at P0-P2, expression of α9L9′T as shown by Cy3-RgIA-5727 label in live tissue explants increased from two to 3 weeks. By 3 weeks post injection 81 Cy3 puncta associated with 57 OHCs were counted in one middle cochlear segment, and 43 Cy3 puncta on 47 OHCs in a second mid-cochlear segment. A few scattered Cy3 puncta were found near IHCs in these tissues, but younger cochleas (when IHCs have numerous efferent synapses) were not examined.

### Example 3

### Noise Induced Hearing Loss

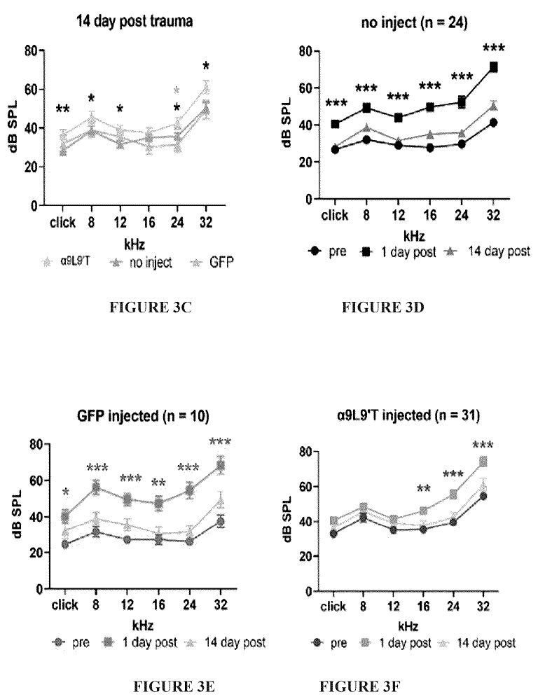

Acoustic trauma causes hearing loss that is greater in α9-null mice than in wild type littermates but is reduced in α9L9′T mice compared to wild type or α9-null. An acoustic trauma protocol was developed to determine if the acoustic vulnerability of α9-null mice could be ‘rescued’ by viral transduction of α9L9′T DNA. It is not possible to know a priori how effective any individual virus injection might be. Thus, to reveal even partial protection, the trauma protocol was designed to produce a temporary threshold shift (20-30 dB across tested frequencies). Mice were exposed to 90 dB ‘white’ noise (2-20 kHz) for 2 hours. ABR thresholds 1- and 14-days post trauma were compared to baseline thresholds collected prior to acoustic trauma. It was noted previously that mice expressing the α9L9′T gain of function nAChR have baseline thresholds 5-10 dB higher than those of wild type littermates. This was attributed to enhanced efferent feedback activated by the test sounds and was reversed by blocking hair cell nAChRs. Mice injected with the α9L9′T-containing virus (combined with GFP-expressing virus) had ABR thresholds averaging 10 dB higher than those of uninjected mice or those given control injections with GFP-expressing virus only (FIG. 3A). There were significant differences in threshold between α9L9′T injected mice and uninjected or GFP injected mice, at all but 12 kHz (multiple unpaired t-tests with Welch correction). Mixed effects linear model testing (restricted maximum likelihood—REML) found that thresholds across all frequencies (including clicks) were significantly elevated in α9L9′T injected compared to uninjected mice (p<0.0001, F (1, 138)=50.09). Likewise, for α9L9′T injected mice compared to GFP injected mice (p<0.0001, F (1, 54)=40.41). Pre-trauma thresholds did not differ between uninjected and GFP injected mice, demonstrating that the injection procedure and viral infection per se were not damaging. At 1 day post acoustic trauma, there were no significant differences in ABR threshold among the three mouse cohorts (FIG. 3B). Thresholds at 14 days post acoustic trauma were again significantly different between α9L9′T injected mice and the others (FIG. 3C), returning to the baseline condition. Mixed effects linear model testing (REML) of the 14-day post trauma thresholds in α9L9′T injected compared to uninjected mice (p<0.0001, F (1, 126)=25.09). Likewise, for α9L9′T injected mice compared to GFP injected mice (p=0.0004, F (1, 269)=12.97). Fourteen days post trauma the original differential sensitivity was re-established.

To further probe these results, each cohort of mice was examined independently for the impact of acoustic trauma at one- and fourteen-days post trauma. In the α9-null mice that received no virus injection (FIG. 3D), the acoustic trauma protocol produced a significant threshold elevation in click and pure tone thresholds from 8 to 32 kHz one day after exposure (delta dB: click 13.8, 8 kHz 17.2, 12 kHz 14.8, 16 Hz 21.9, 24 kHz 22.7, 32 kHz 28.5, p<0.001 for all frequencies, Welch correction for multiple unpaired t-tests). After 14 days, ABR threshold returned to near baseline, thus, a temporary threshold shift. Mice injected with the GFP-containing virus only (FIG. 3E) experienced threshold shifts at one day post trauma similar to those in the un-injected mice, with recovery at 14 days (delta dB: click 15.8, 8 kHz 24.7, 12 kHz 22.5, 16 kHz 20.2, 24 kHz 28.5, 32 kHz 30.8; p<0.01 or 0.001 for comparisons at each frequency (clicks p<0.05), Welch correction for multiple unpaired t-tests). In contrast, mice that received a unilateral injection of the α9-containing virus (plus the GFP-containing virus) had no, or smaller shifts at all frequencies one day post trauma, with significant hearing loss only at mid and upper frequencies (FIG. 3F). Threshold shifts were (dB): 8.7, 8 kHz 8.9, 12 kHz 7.4, 16 kHz 12.1, 24 kHz 16.8, 32 kHz 19.5; significant for 16, 24 and 32 kHz only. Mixed effects linear model testing (REML) was run to compare threshold shifts at one day post trauma between experimental conditions and across frequencies. The one-day threshold shifts in mice injected with α9-containing virus (plus GFP-containing virus) were significantly less than those for control mice that received no injection (p=0.0013, F (1, 30.00)=12.67). A significant difference in threshold shift also was found comparing α9L9′T (plus GFP-containing virus) injected mice to those injected with the GFP-containing virus only (p<0.0001, F (1, 231)=28.28). Thus, injection of an AAV vector expressing the gain of function α9L9′T nAChR subunit specifically reduced the temporary threshold shift caused by acoustic trauma in α9-null mice.

### Example 4

### Distortion Product Oto-Acoustic Emissions

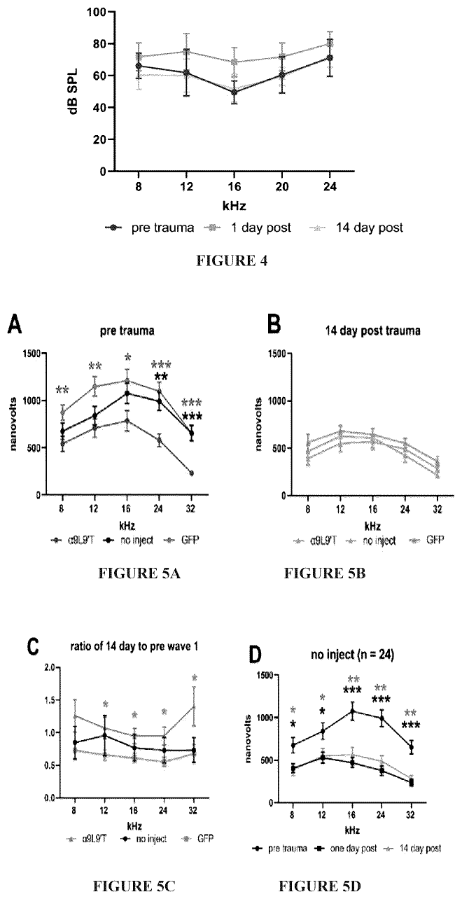

The impact of acoustic trauma on distortion product otoacoustic emissions was examined in mice injected with the α9L9′T gain of function virus. One day after acoustic trauma there appeared to be some threshold elevation, however that change did not rise to significance for any frequency tested (FIG. 4) (multiple t-tests with Welch correction and two-way ANOVA). Thus, an acoustic trauma protocol that reliably elevated ABR thresholds at all frequencies in control mice (FIG. 3) did not alter DPOAE thresholds significantly in the gain-of-function transduced mice.

### Example 5

### ABR Waveforms

A change in ABR threshold could be due to degraded mechano-transduction, reduced outer hair cell function, altered endolymph, etc. Damage also can occur at the synaptic contacts between inner hair cells and type I afferents without necessarily altering acoustic thresholds. This deficit can be revealed by measuring the amplitude and latency of wave 1 of the ABR. Acoustic trauma causes a reduction in maximum amplitude, indicating fewer type I afferents contributing to the initial compound action potential, either from reduced numbers, or a spread in latencies. A9L9′T knock-in mice have reduced amplitude wave 1 of the ABR compared to wildtype or α9 knockout mice, and this was unchanged after acoustic trauma. Likewise, in the present work, the baseline (pre-trauma) amplitude of wave 1 of the ABR was significantly smaller in mice injected with α9-containing virus (plus GFP virus) than in mice that were not injected, as well as that of mice injected with GFP-containing virus only (FIG. 5A). Two-way ANOVA for α9L9′T injected versus uninjected gave p<0.0001, F (1, 298)=33.38. Likewise, for α9L9′T injected versus GFP injected, p<0.0001, F (1, 269)=51.93. ABR wave 1 of GFP injected mice versus that of uninjected mice was not significantly different across frequencies (p=0.2578, F (1, 245)=1.286). At 14 days after acoustic trauma there were no significant differences at each frequency among all cohorts (multiple unpaired t-tests with Welch correction) (FIG. 5B). Further insight is obtained by examining each cohort separately.

One day after acoustic trauma both groups of control mice (non-injected and GFP-injected) displayed a significant reduction in the amplitude of wave 1 of the ABR evoked by the loudest clicks and pure tones from 8 to 32 kHz (FIGS. 5D, 5E). Wave 1 amplitude remained smaller 14 days after acoustic trauma, indicating that this deficit did not recover (unlike elevated ABR threshold). Wave 1 of the ABR of mice that received the α9L9′T-containing virus also was reduced after acoustic trauma but recovered substantially by 14 days (FIG. 5F) (mixed effects model (REML) for day 14 compared to day 1 amplitude; p=0.0019, F (1, 150)=9.997). This is also evident when comparing the ratios of wave 1 amplitudes at 14 days to the original pre-trauma value (FIG. 5C). A mixed-effects model (REML) for the α9L9′T injected mice showed that the ratio of wave 1 amplitudes at 14 days to the pre-trauma value was significantly larger than that of the GFP injected mice (p<0.0001, F (1, 261)=20.40) and that of the uninjected mice (p=0.0116, F (1, 261)=6.469). Thus, mice in all conditions underwent significant wave 1 amplitude loss one day post acoustic trauma. However, the α9L9′T injected mice significantly recovered this parameter while both sets of control mice did not.

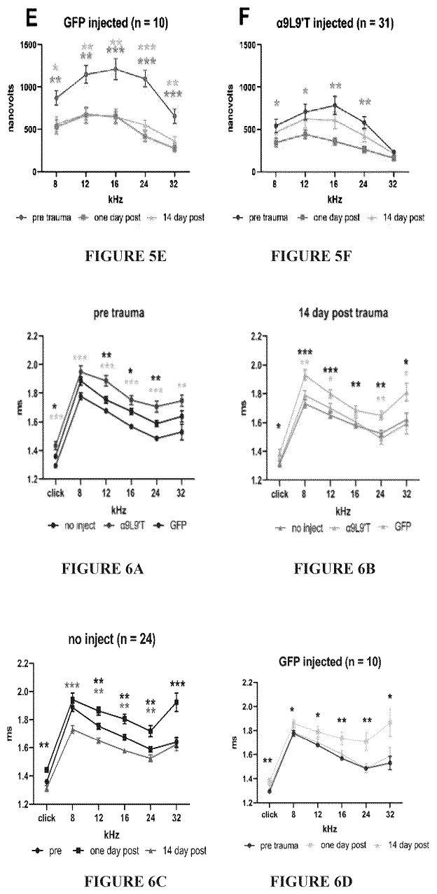

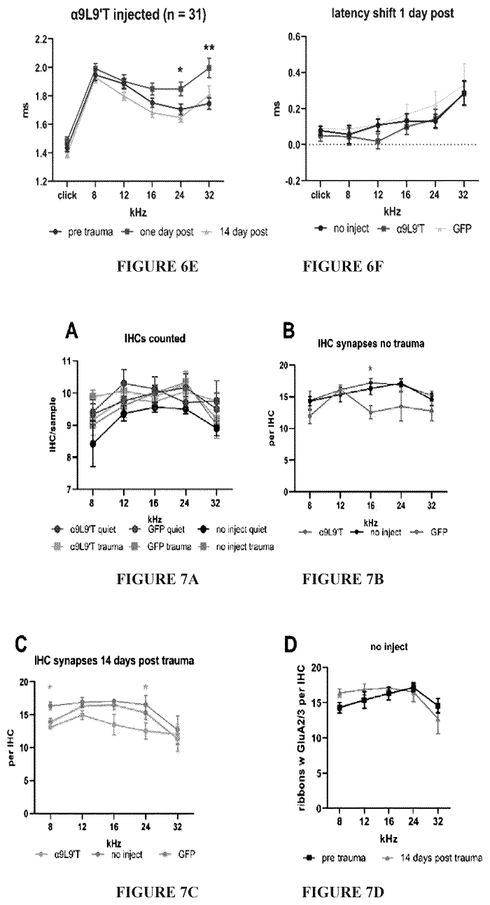

The latency to wave 1 of the ABR is another indicator of afferent excitation. Longer latencies suggest disruption in coordinate firing or otherwise delayed conduction. This disruption could be pre- or postsynaptic in origin. ABR wave 1 latency was significantly greater in α9L9′T injected mice than in uninjected, or GFP injected mice (FIG. 6A). Fourteen days post acoustic trauma wave 1 latency remained significantly longer in α9L9′T injected than in both sets of control mice (FIG. 6B). Wave 1 latency was significantly greater one day after acoustic trauma in control cohorts: uninjected control mice (FIG. 6C), GFP injected mice (FIG. 6E). In contrast, ABR wave 1 latencies increased only at 24 and 32 kHz in α9L9′T injected mice (FIG. 6D). However, direct comparison of the latency shift at one day post trauma showed no significant differences in the extent of latency shift across frequency among all groups (FIG. 6F). Latencies recovered to pre-trauma control values at 14 days in all groups.

### Example 6

### Ribbon Synapses of IHCs

Changes in wave 1 of the ABR have been correlated with the loss of synaptic contacts between IHCs and type I afferents. Thus, ribbon synapses were counted in IHCs of α9 knockout mice with or without acoustic trauma (2-hour exposure to 90 dB broadband sound), with or without viral transduction (either α9L9′T virus plus GFP virus, or GFP virus alone this latter serving as injection control condition). This generated six experimental groups for analysis: 1. No virus injection, no acoustic trauma, 2. No virus injection plus acoustic trauma, 3. GFP viral injection, no acoustic trauma, 4. GFP virus injection plus acoustic trauma, 5.A9L9′T and GFP virus injection, no acoustic trauma, 6. A9L9′T and GFP virus plus acoustic trauma. Synaptic immunohistology was carried out 14 or 15 days after acoustic trauma (postnatal day 45-46).

Each cochlea was divided into segments of approximately 100 μm in length (containing on average 10 IHCs and 30 OHCs), at locations corresponding to center frequencies of 8, 12, 16, 24 and 32 kHz (based on the standardized mouse cochlear frequency map (Muller, von Hunerbein et al. 2005). CtBP2 (ribbons), GluA2/3 (postsynaptic AMPAR clusters), ribbons colocalized with GluA2/3 puncta, and synaptophysin (efferent terminals) immunopuncta were counted in confocal z-stacks. Counts were conducted repeatedly using ImageJ and Imaris by blinded observers and consolidated post hoc before identifying experimental condition. Ribbons per IHC ranged from 6 to 18 depending on cochlear position, trending to highest numbers at the 16 kHz position in control cochleas. Immunolabel for GluA2/3 provided the lowest signal to noise. Therefore, the better-labeled ribbons were first demarcated in each tissue sample, then the GluA2/3 channel was examined for corresponding immunopuncta. In some cases, the merged colors also were used. Overall, 5% of ribbons could not be associated with GluA2/3.

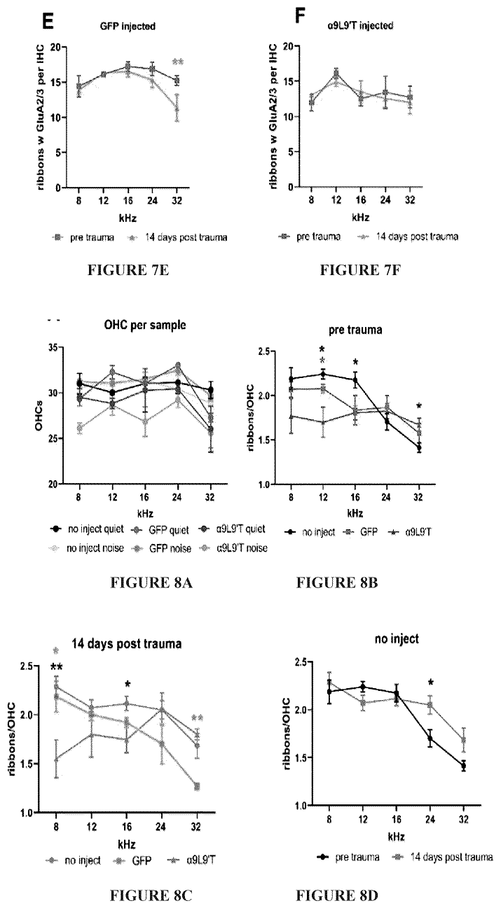

Synapses per IHC ranged from 6 to 18 depending on cochlear position, tending to highest numbers at the 16 or 24 kHz positions in control cochleas. Inner hair cells from α9L9′T injected cochleas had fewer synapses, a peak at 12 kHz but a flatter tonotopic distribution overall (FIG. 7B). Synapse counts in IHCs of α9L9′T injected cochleas differed from controls (p=0.008, F(2, 60)=5.232), but the interaction with frequency failed significance (p=0.699, F (8, 60)=0.690). Following acoustic trauma (FIG. 7C), α9L9′T injected IHCs had significantly fewer synapses than controls (p=0.0008, F(2,64) but the interaction with frequency remained non-significant (p=0.359, F(8,64)=1.124). The effect of acoustic trauma on synapse numbers within each cohort revealed almost no loss with the exception of the 32 kHz position in GFP injected cochleas. There was no synapse loss at 32 kHz in the α9L9′T injected cochleas, perhaps corresponding with the better recovery of wave 1 (FIG. 5F). Overall, however, these data on synapse number do not correlate with the sustained drop in amplitude of wavel of the ABR for all frequencies in control or experimental cochleas.

### Example 7

### Afferent and Efferent Synapses of OHCs

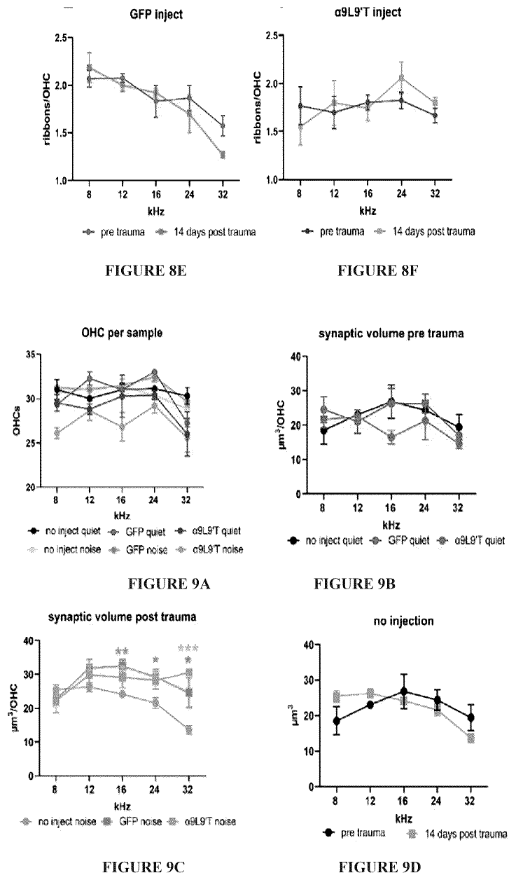

Acoustic trauma has opposing effects on ribbon synapses of inner and outer hair cells, decreasing those of inner hair cells but increasing those of outer hair cells. Thus, reduced acoustic trauma in α9L9′T-injected mice might be expected to prevent ribbon augmentation in outer hair cells. There were no significant differences in number of outer hair cells among all cohorts. However, there was some effect of the α9L9′T treatment on outer hair cell ribbons. In uninjected and GFP injected cochleas there was a falling gradient of ribbons per outer hair cells (i.e., more in apical outer hair cells, fewer in basal outer hair cells) as one moved from lower to higher frequency positions in the cochlea, while α9L9′T injected cochleas had equal numbers of outer hair cell ribbons at all locations (FIG. 8B). After acoustic trauma, these differential patterns were more pronounced with α9L9′T injected cochleas developing a tonotopic gradient that appeared to reverse the control pattern (FIG. 8C). Linear mixed-model testing showed that the effect of noise differed significantly by condition (p=0.0047, F(2,60)=5.86) and frequency (p=0.0059, F(4,60)=4.025), reflecting these differing tonotopic patterns of outer hair cell ribbons.

While between group comparisons were significant for the effect of noise, tonotopic gradients dominated within group ribbon counts. For uninjected cochleas (FIG. 8D), two-way ANOVA showed that frequency (cochlear location) accounted for 42% of the total variance, p<0.0001, F (4, 67)=16.77. But, pre-versus post-noise exposure was not significant, p=0.1173, F=2.517. Two-way ANOVA for GFP injected cochleas (FIG. 8E) showed that frequency (cochlear location) accounted for 46.54% of the total variance, with p=0.0002, F (4, 31)=7.95. Synapse counts pre-versus post-noise exposure were not significantly different, p=0.429, F=0.64. α9L9′T injected mice differed from the control tonotopic pattern both pre- and post-acoustic trauma (FIG. 8F). This did not rise to statistical significance as a function of acoustic trauma or frequency. Two-way ANOVA for α9L9′T injected cochleas showed that frequency (cochlear location) accounted for only 9.9% of the total variance, with p=0.475, F (4, 30)=0.90 with no significant difference after noise exposure, p=0.699, F (1,30)=0.1524. Thus, while α9L9′T injected cochleas lost the control tonotopic pattern of outer hair cell ribbon expression, noise exposure, as for the control cochleas, did not alter that pattern significantly.

### Example 8

### Efferent Synapses on Outer Hair Cells

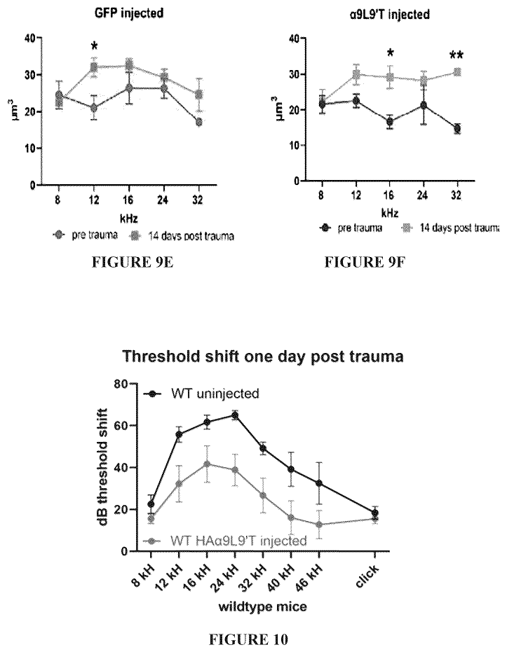

Efferent innervation density of OHCs in α9 transgenic mouse cochleas with total presynaptic terminal volume (sum of all contacts per OHC) are smaller on α9 null OHCs and larger on OHCs of α9L9′T gain-of-function mice. This issue and the impact of noise were examined by quantifying efferent synaptic volumes on OHCs in cochlear tissue of control and α9L9′T injected mice with and without acoustic trauma. Efferent synaptic volumes on individual OHCs ranged between 10 and 40 μm³ with average values between 15 and 25 μm³ (FIG. 9B) with a trend to lower values at the 32 kHz position. There were no significant differences among all cohorts before noise exposure (two-way ANOVA) (FIG. 9B). Average values were significantly higher in both sets of viruses injected cochleas from noise exposed mice, compared to uninjected (FIG. 9C, both as a function of frequency, p=0.0097, and condition, p<0.0001, two-way ANOVA). Examining efferent synaptic volumes in each cohort independently found no effect of noise exposure on efferent volumes of OHCs in uninjected cochleas (FIG. 9D) and a significant difference only at 12 kHz for GFP injected cochleas (FIG. 9E). However, efferent synaptic volumes were significantly larger in α9L9′T injected cochleas after noise exposure (FIG. 9F, effect of noise p=0.0004, two-way ANOVA, F(1,31)=15.94).

### Example 9

### Discussion

The present work explored a ‘gene rescue’ strategy using viral transduction of the gain of function α9L9′T to enhance acoustic protection in α9-null mice. The AAV2.7m8 virus was shown to transduce cochlear hair cells efficiently with green fluorescent protein (GFP). Virally mediated expression of α9L9′T at synaptic sites in α9-null hair cells 3 weeks post injection was demonstrated in the present work by labeling with Cy3-RgIA-5727, a fluorophore-conjugated modification of a Conus venom peptide that is a high affinity, slowly reversible antagonist of α9-containing nAChRs.

Surgical introduction of virus was not damaging per se, as shown by equivalent hearing in mice receiving a GFP-containing virus compared to uninjected mice. Rather, injection of α9L9′T containing virus specifically altered ABR and DPOAE thresholds and sensitivity to acoustic trauma. The acoustic protection was not as effective as in the α9L9′T knock-in, as might be expected for this exploratory study. But injection with α9L9′T-containing virus did mitigate the usual pattern of noise induced threshold shifts, completely preserving ABR threshold at lower frequencies, and reducing the loss at higher frequencies. A counter argument proposes that α9L9′T transduced mice were simply less sensitive to sound, so the smaller threshold shift results from a higher initial baseline. In other words, α9L9′T transduction is equivalent to putting in ear plugs. However, efferent feedback is arguably quite different from ear plugging. Efferent neurons have sensitive, V-shaped tuning curves to sound, providing protection that is both intensity and frequency tuned. As sound gets louder, efferents fire more frequently, transmitter release facilitates, suppression gets stronger, and is directed specifically against the frequency content of that loud sound. Additional support is derived from studies on α9L9′T knock in mice. Efferent synapses in these mice have a significantly lower resting probability of release than in wildtype but facilitate strongly with repetitive activation. This is thought to be a form of homeostatic plasticity to compensate for the stronger postsynaptic effect of α9L9′T nAChRs. The benefit for hearing is that efferent activity will be still less effective near threshold (than in wildtype) and even more effective for louder sounds.

Does gene rescue preserve the ABR waveform and alter synapse numbers? Following acoustic trauma, all cohorts of mice experienced a 50% drop in wave 1 ABR amplitude that recovered 14 days later only in the α9L9′T injected mice. Based on this result, and the recovery of ABR threshold in these same animals, this suggests that this type of acoustic trauma caused a temporary threshold shift, but lasting ‘hidden hearing loss,’ i.e., reduced numbers of responding type I afferents for a saturating loud sound in the control animals only. Recovery of wave 1 amplitude in α9L9′T injected mice suggests that synaptopathy also was mitigated. Wave 1 latency measurements partially followed this pattern, with a smaller increase in latency for α9L9′T injected mice than in either control cohort. However, in contrast to wave 1 amplitude that remained suppressed 14 days post trauma, latencies recovered completely in all cohorts of mice. Inner hair cell synapse counts 14 days post trauma did not cohere completely with the ABR waveform changes. In uninjected and GFP injected cochleas acoustic trauma reduced synapse numbers at 32 kHz only, while wave 1 amplitude remained suppressed for all frequencies. On the other hand, α9L9′T injected cochleas lost no synapses 14 days after trauma, consistent with recovery of ABR wave 1 in these mice. There is a qualitative suggestion that the tonotopic distribution of ribbon synapses of inner hair cells was altered in α9L9′T injected cochleas, appearing to be flatter than the peaked distribution of control cochleas. Alteration of tonotopic distribution was more obvious for ribbons in outer hair cells. Uninjected and GFP injected cochleas have a significantly declining gradient of ribbon numbers moving from lower to higher frequency positions. This tonotopic gradient is not present, and in fact reverses after trauma in α9-injected cochleas. Thus, the difference in ribbon counts for outer hair cells at low frequency positions was even greater among cohorts after trauma, perhaps because the α9L9′T-injected mice were better protected and so were not driven to increase outer hair cell ribbon synapses as reported previously for more severe acoustic trauma. In the present work, efferent synapses on outer hair cells also show some influence of the α9L9′T virus. After acoustic trauma, α9-injected cochleas had significantly larger efferent synaptic contacts. Neither uninjected nor GFP-injected cochleas showed significant differences after trauma.

Comparison to transgenic mice. How similar was viral transduction to genetic knock in of α9L9′T? Baseline thresholds in the virally transduced mice were on average 10 dB higher than those of control mice. Thresholds of α9L9′T knock in were 5 to 10 dB higher than wildtype. The knock in mice were completely protected from acoustic trauma, whereas viral transduction was only partially protective, suggesting less efficient expression compared to the knock in. Work on α9L9′T knock in mice showed that inner hair cell synaptopathy (hidden hearing loss) also was ameliorated compared to knockout or wildtype mice. The amplitude of wave 1 was irreversibly reduced by trauma in wildtype and α9 knockout mice, but unchanged in the α9L9′T knock in. In the present work, ABR wave 1 amplitude was reduced by trauma in all cohorts but recovered only in those mice virally transduced with α9L9′T. Thus, viral expression of α9L9′T has some benefit for synaptopathy as well. However, caution is required since wave 1 amplitude was initially smaller, and latencies longer for α9L9′T-injected mice compared to uninjected or GFP-injected mice. There were no significant differences in the effect of trauma on wave 1 latency among these three cohorts. In α9L9′T transgenic mice inner hair cells maintain and actually increase synaptic counts after acoustic trauma, whereas wildtype and knockout inner hair cells lost synapses. Rather, reduction of ABR wave 1 amplitude was not reflected in a comparable change of inner hair cell synapses in any of the cohorts of mice.

Conclusions. A9L9′T viral transduction will be tested in wildtype mice. Behavioral thresholds need to be tested in more complex acoustic environments to learn if acoustic protection after viral transduction improves discriminative hearing. The longevity of transfected DNA needs to be explored further. Studies in mice suggest continued expression for months. Finally, while there is no evidence for altered vestibular function, α9-containing nAChRs do mediate inhibition of type II vestibular hair cells, whose consequences should be addressed. The present results constitute a proof-of-principle to motivate further exploration for therapeutic application. Age-related hearing loss is worsened by genetic variants that associate with earlier onset and is exacerbated by noise exposure. Genetic markers and family history of hearing loss can identify an initial population likely to benefit most from enhanced efferent protection. Such a strategy would provide still more general benefits. Long-lasting enhancement of efferent protection could play a role wherever acoustic exposure exacerbates hearing loss, whether monogenic, environmental, or of undefined etiology. It could be complementary to gene replacement for monogenic deafness. Long-lasting, virally mediated α9L9′T expression could reduce the dosage or frequency of small molecule therapies (e.g., prophylaxis for ototoxic medications, or positive allosteric modulators of the nAChR). Ultimately one could imagine efferent upregulation as generally recommended for amelioration of age-related hearing loss, especially for those with unavoidable exposure to damaging levels of sound in the workplace or military service. A compelling argument for this strategy is that efferent activity is itself regulated by the acoustic environment. Thus, genetic enhancement of efferent inhibition leverages an intrinsic protective mechanism of the inner ear. Enhanced efferent feedback also might benefit gain of function pathologies such as hyperacusis or tinnitus. Therefore, the present study pursues a genetic ‘gain-of-function’ strategy using viral transduction to repair the inner ear's own protective feedback and thereby reduce acoustic trauma. An additional benefit of this strategy is that it could apply to any pathology that is exacerbated by noise, and so would be complementary to other therapies. This could have particular benefit for those at risk of early onset, activity dependent hearing loss.

While important questions remain, the results presented above constitute a proof-of-principle to motivate further exploration for therapeutic application. Age-related hearing loss is worsened by genetic variants that associate with earlier onset and is exacerbated by noise exposure. Genetic markers and family history of hearing loss can identify an initial population likely to benefit most from enhanced efferent protection. Such a strategy could provide still more general benefits. Long-lasting enhancement of efferent protection could play a role wherever acoustic exposure exacerbates hearing loss, whether genetic, environmental, or of undefined etiology. It could be complementary to gene replacement for monogenic deafness. Long-lasting, virally mediated α9L9′T expression could reduce the dosage or frequency of small molecule therapies (e.g., prophylaxis for ototoxic medications, or positive allosteric modulators of the nAChR). Ultimately one could imagine efferent upregulation as generally recommended for amelioration of age-related hearing loss, especially for those with unavoidable exposure to damaging levels of sound in the workplace or military service. A compelling argument for this strategy is that efferent activity is itself regulated by the acoustic environment. Thus, genetic enhancement of efferent inhibition leverages an intrinsic protective mechanism of the inner ear. An interesting associated question is whether enhanced efferent feedback also might benefit gain of function pathologies such as hyperacusis or tinnitus.

### Example 10

### Virally Expressed Gain-of-Function nACHRs in Cochlear Hair Cells of Wildtype Mice Reduces Acoustic Trauma

Studies on nAChR knock-out and gain-of-function knock-in mice have confirmed that the strength of efferent cholinergic inhibition correlates with protection against acoustic trauma (Taranda et al., 2009, Boero et al., 2020). This effect has been replicated by viral gene therapy: ‘rescuing’ nAChR knock-out mice by viral transduction of the gain of function nAChR (α9L9′T) (Zhang et al., 2023, and Examples 1-9 above).

A further study has been performed to analyze the effect in wildtype animals. It has been shown that a similar effect can be demonstrated in wildtype mice in which the virally produced gain of function nAChR combines with native nAChRs. Using an HA-tagged α9L9′T nAChR robust expression was found postsynaptic to efferent terminals on outer hair cells (data not shown). Mice injected with the virus expressing HA-tagged α9L9′T nAChR showed enhanced protection against acoustic trauma (FIG. 10).

Virally expressed HA-tagged nAChRs were expressed in wildtype outer hair cells. An AAV was injected into the inner ear to drive expression. HA immunolabel revealed clustered HA-nAChRs in outer hair cells aligned with presynaptic efferent terminals immunolabeled for synaptic vesicles. As illustrated in FIG. 10, acoustic trauma caused a hearing loss in WT animals (seen as an elevation of the threshold for the auditory brainstem response-ABR). The shift was substantially less in mice that had been injected with a virus expressing HA-tagged α9L9′T nAChRs, thereby demonstrating the protective effect against acoustic trauma in WT animals.

### REFERENCES

Ahmed, H., O. Shubina-Oleinik and J. R. Holt (2017). “Emerging Gene Therapies for Genetic Hearing Loss.” J Assoc Res Otolaryngol 18(5): 649-670.
Akil, O., F. Dyka, C. Calvet, A. Emptoz, G. Lahlou, S. Nouaille, J. Boutet de Monvel, J. P. Hardelin, W. W. Hauswirth, P. Avan, C. Petit, S. Safieddine and L. R. Lustig (2019). “Dual AAV-mediated gene therapy restores hearing in a DFNB9 mouse model.” Proc Natl Acad Sci U S A 116(10): 4496-4501.
Akil, O., R. P. Seal, K. Burke, C. Wang, A. Alemi, M. During, R. H. Edwards and L. R. Lustig (2012). “Restoration of hearing in the VGLUT3 knockout mouse using virally mediated gene therapy.” Neuron 75(2): 283-293.
Al-Moyed, H., A. P. Cepeda, S. Jung, T. Moser, S. Kugler and E. Reisinger (2019). “A dual-AAV approach restores fast exocytosis and partially rescues auditory function in deaf otoferlin knock-out mice.” EMBO Mol Med 11(1).
Ballestero, J., J. Zorrilla de San Martin, J. Goutman, A. B. Elgoyhen, P. A. Fuchs and E. Katz (2011). “Short-term synaptic plasticity regulates the level of olivocochlear inhibition to auditory hair cells.” J Neurosci 31(41): 14763-14774.
Bennett, J. (2000). “Gene therapy for retinitis pigmentosa.” Curr Opin Mol Ther 2(4): 420-425.
Boero, L. E., V. C. Castagna, M. N. Di Guilmi, J. D. Goutman, A. B. Elgoyhen and M. E. Gomez-Casati (2018). “Enhancement of the Medial Olivocochlear System Prevents Hidden Hearing Loss.” J Neurosci 38(34): 7440-7451.
Boero, L. E., V. C. Castagna, G. Terreros, M. J. Moglie, S. Silva, J. C. Maass, P. A. Fuchs, P. H. Delano, A. B. Elgoyhen and M. E. Gomez-Casati (2020). “Preventing presbycusis in mice with enhanced medial olivocochlear feedback.” Proc Natl Acad Sci U S A 117(21): 11811-11819.
Brown, M. C. (1987). “Morphology of labeled efferent fibers in the guinea pig cochlea.” J Comp Neurol 260(4): 605-618.
Canlon, B., A. Fransson and A. Viberg (1999). “Medial olivocochlear efferent terminals are protected by sound conditioning.” Brain Res 850(1-2): 253-260.
Dalkara, D., L. C. Byrne, R. R. Klimczak, M. Visel, L. Yin, W. H. Merigan, J. G. Flannery and D. V. Schaffer (2013). “In vivo-directed evolution of a new adeno-associated virus for therapeutic outer retinal gene delivery from the vitreous.” Sci Transl Med 5(189): 189ra176.
Delmaghani, S. and A. El-Amraoui (2020). “Inner Ear Gene Therapies Take Off: Current Promises and Future Challenges.” J Clin Med 9(7).
Di Guilmi, M. N., L. E. Boero, V. C. Castagna, A. Rodriguez-Contreras, C. Wedemeyer, M. E. Gomez-Casati and A. B. Elgoyhen (2019). “Strengthening of the Efferent Olivocochlear System Leads to Synaptic Dysfunction and Tonotopy Disruption of a Central Auditory Nucleus.” J Neurosci 39(36): 7037-7048.
Elgoyhen, A. B. (2022). “The alpha9alpha10 nicotinic acetylcholine receptor: a compelling drug target for hearing loss?” Expert Opin Ther Targets: 1-12.
Elgoyhen, A. B., E. Katz and P. A. Fuchs (2009). “The nicotinic receptor of cochlear hair cells: a possible pharmacotherapeutic target?” Biochem Pharmacol 78(7): 712-719.
Ellison, M., C. Haberlandt, M. E. Gomez-Casati, M. Watkins, A. B. Elgoyhen, J. M. McIntosh and B. M. Olivera (2006). “Alpha-RgIA: a novel conotoxin that specifically and potently blocks the alpha9alpha10 nAChR.” Biochemistry 45(5): 1511-1517.
Fisher, F., Y. Zhang, P. F. Y. Vincent, J. Gajewiak, T. J. Gordon, E. Glowatzki, P. A. Fuchs and J. M. McIntosh (2021). “Cy3-RgIA-5727 Labels and Inhibits alpha9-Containing nAChRs of Cochlear Hair Cells.” Front Cell Neurosci 15: 697560.
Frank, M. M. and L. V. Goodrich (2018). “Talking back: Development of the olivocochlear efferent system.” Wiley Interdiscip Rev Dev Biol 7(6): e324.
Fuchs, P. A. and A. M. Lauer (2019). “Efferent Inhibition of the Cochlea.” Cold Spring Harb Perspect Med 9(5).
Fuente, A. (2015). “The olivocochlear system and protection from acoustic trauma: a mini literature review.” Front Syst Neurosci 9: 94. Galambos, R. (1956). “Suppression of auditory nerve activity by stimulation of efferent fibers to cochlea.” J Neurophysiol 19(5): 424-437.
Gates, G. A., P. Schmid, S. G. Kujawa, B. Nam and R. D'Agostino (2000). “Longitudinal threshold changes in older men with audiometric notches.” Hear Res 141(1-2): 220-228.
Guinan, J. J., Jr. (2010). “Cochlear efferent innervation and function.” Curr Opin Otolaryngol Head Neck Surg 18(5): 447-453.
Hashimoto, K., T. T. Hickman, J. Suzuki, L. Ji, D. C. Kohrman, G. Corfas and M. C. Liberman (2019). “Protection from noise-induced cochlear synaptopathy by virally mediated overexpression of NT3.” Sci Rep 9(1): 15362.
Hultcrantz, M. and H. S. Li (1993). “Inner ear morphology in CBA/Ca and C57BL/6J mice in relationship to noise, age and phenotype.” Eur Arch Otorhinolaryngol 250(5): 257-264.
Isgrig, K., D. S. McDougald, J. Zhu, H. J. Wang, J. Bennett and W. W. Chien (2019). “AAV2.7m8 is a powerful viral vector for inner ear gene therapy.” Nat Commun 10(1): 427.
Jimenez, J. E., A. Nourbakhsh, B. Colbert, R. Mittal, D. Yan, C. L. Green, E. Nisenbaum, G. Liu, N. Bencie, J. Rudman, S. H. Blanton and X. Zhong Liu (2020). “Diagnostic and therapeutic applications of genomic medicine in progressive, late-onset, nonsyndromic sensorineural hearing loss.” Gene 747: 144677.
Klinke, R. and N. Galley (1974). “Efferent innervation of vestibular and auditory receptors.” Physiol Rev 54(2): 316-357.
Lauer, A. M., S. V. Jimenez and P. H. Delano (2021). “Olivocochlear efferent effects on perception and behavior.” Hear Res: 108207.
Lauer, A. M. and B. J. May (2011). “The medial olivocochlear system attenuates the developmental impact of early noise exposure.” J Assoc Res Otolaryngol 12(3): 329-343.
Li, H. S., M. Hultcrantz and E. Borg (1993). “Influence of age on noise-induced permanent threshold shifts in CBA/Ca and C57BL/6J mice.” Audiology 32(3): 195-204.
Liberman, M. C. and M. C. Brown (1986). “Physiology and anatomy of single olivocochlear neurons in the cat.” Hear Res 24(1): 17-36.
Liberman, M. C., L. W. Dodds and S. Pierce (1990). “Afferent and efferent innervation of the cat cochlea: quantitative analysis with light and electron microscopy.” J Comp Neurol 301(3): 443-460.
Liberman, M. C. and S. G. Kujawa (2017). “Cochlear synaptopathy in acquired sensorineural hearing loss: Manifestations and mechanisms.” Hear Res 349:138-147.
Liberman, M. C., L. D. Liberman and S.F. Maison (2014). “Efferent feedback slows cochlear aging.” J Neurosci 34(13): 4599-4607.
Lina, I. A. and A. M. Lauer (2013). “Rapid measurement of auditory filter shape in mice using the auditory brainstem response and notched noise.” Hear Res 298:73-79.
Luebke, A. E. and P. K. Foster (2002). “Variation in inter-animal susceptibility to noise damage is associated with alpha 9 acetylcholine receptor subunit expression level.” J Neurosci 22(10): 4241-4247.
Lustig, L. and O. Akil (2019). “Cochlear Gene Therapy.” Cold Spring Harb Perspect Med 9(9).
Maison, S. F., A. E. Luebke, M. C. Liberman and J. Zuo (2002). “Efferent protection from acoustic injury is mediated via alpha9 nicotinic acetylcholine receptors on outer hair cells.” J Neurosci 22(24): 10838-10846.
Maison, S. F., H. Usubuchi and M. C. Liberman (2013). “Efferent feedback minimizes cochlear neuropathy from moderate noise exposure.” J Neurosci 33(13): 5542-5552.
Muller, M., K. von Hunerbein, S. Hoidis and J. W. Smolders (2005). “A physiological place-frequency map of the cochlea in the CBA/J mouse.” Hear Res 202(1-2): 63-73.
Murthy, V., J. Taranda, A. B. Elgoyhen and D. E. Vetter (2009). “Activity of nAChRs containing alpha9 subunits modulates synapse stabilization via bidirectional signaling programs.” Dev Neurobiol 69(14): 931-949.
Nadol, J. B., Jr. and B. J. Burgess (1994). “Supranuclear efferent synapses on outer hair cells and Deiters' cells in the human organ of Corti.” Hear Res 81(1-2): 49-56.
Nouvian, R., M. Eybalin and J. L. Puel (2015). “Cochlear efferents in developing adult and pathological conditions.” Cell Tissue Res 361(1): 301-309.
Prosen, C. A., K. G. Bath, D. E. Vetter and B. J. May (2000). “Behavioral assessments of auditory sensitivity in transgenic mice.” J Neurosci Methods 97(1): 59-67.
Rajan, R. and B. M. Johnstone (1988). “Binaural acoustic stimulation exercises protective effects at the cochlea that mimic the effects of electrical stimulation of an auditory efferent pathway.” Brain Res 459(2): 241-255.
Robertson, D. and M. Gummer (1985). “Physiological and morphological characterization of efferent neurons in the guinea pig cochlea.” Hear Res 20(1): 63-77.
Roux, I., E. Wersinger, J. M. McIntosh, P. A. Fuchs and E. Glowatzki (2011). “Onset of cholinergic efferent synaptic function in sensory hair cells of the rat cochlea.” J Neurosci 31(42): 15092-15101.
Sato, M., M. M. Henson and D. W. Smith (1997). “Synaptic specializations associated with the outer hair cells of the Japanese macaque.” Hear Res 108(1-2): 46-54.
Stamataki, S., H. W. Francis, M. Lehar, B. J. May and D. K. Ryugo (2006). “Synaptic alterations at inner hair cells precede spiral ganglion cell loss in aging C57BL/6J mice.” Hear Res 221(1-2): 104-118.
Taranda, J., S. F. Maison, J. A. Ballestero, E. Katz, J. Savino, D. E. Vetter, J. Boulter, M. C. Liberman, P. A. Fuchs and A. B. Elgoyhen (2009). “A point mutation in the hair cell nicotinic cholinergic receptor prolongs cochlear inhibition and enhances noise protection.” PloS Biol 7(1): e18.
Vetter, D. E., E. Katz, S. F. Maison, J. Taranda, S. Turcan, J. Ballestero, M. C. Liberman, A. B. Elgoyhen and J. Boulter (2007). “The alpha10 nicotinic acetylcholine receptor subunit is required for normal synaptic function and integrity of the olivocochlear system.” Proc Natl Acad Sci U S A 104(51): 20594-20599.
Vyas, P., M. B. Wood, Y. Zhang, A. C. Goldring, F. Z. Chakir, P. A. Fuchs and H. Hiel (2020). “Characterization of HA-tagged alpha9 and alpha10 nAChRs in the mouse cochlea.” Sci Rep 10(1): 21814.
Wang, J. and J. L. Puel (2020). “Presbycusis: An Update on Cochlear Mechanisms and Therapies.” J Clin Med 9(1).
Wang, Y., M. Sanghvi, A. Gribizis, Y. Zhang, L. Song, B. Morley, D. G. Barson, J. Santos-Sacchi, D. Navaratnam and M. Crair (2021). “Efferent feedback controls bilateral auditory spontaneous activity.” Nat Commun 12(1): 2449.
Wood, M. B., N. Nowak, K. Mull, A. Goldring, M. Lehar and P. A. Fuchs (2021). “Acoustic Trauma Increases Ribbon Number and Size in Outer Hair Cells of the Mouse Cochlea.” J Assoc Res Otolaryngol 22(1): 19-31.
Wu, J., P. Solanes, C. Nist-Lund, S. Spataro, O. Shubina-Oleinik, I. Marcovich, H. Goldberg, B. L. Schneider and J. R. Holt (2020). “Single and Dual Vector Gene Therapy with AAV9-PHP.B Rescues Hearing in Tmc1 Mutant Mice.” Mol Ther.
Wu, J. S., E. Yi, M. Manca, H. Javaid, A. M. Lauer, and E. Glowatzki (2020). “Sound exposure dynamically induces dopamine synthesis in cholinergic LOC efferents for feedback to auditory nerve fibers.” Elife 9.
Zhang Y Y, Hiel H, Vincent P F Y, Wood M B, Elgoyhen A B, Chien W, Lauer A and Fuchs P A. 2023. Engineering olivocochlear inhibition to reduce acoustic trauma. Molecular Therapy Methods and Clinical Development 29:17-31. DOI: 10.1016/j.omtm.2023.02.011, PMID: 36941920 PMCID: PMC10023855

Although the invention has been described with reference to the above examples, it will be understood that modifications and variations are encompassed within the spirit and scope of the invention. Accordingly, the invention is limited only by the following claims.

1. A method of preventing hearing loss in a subject comprising administering to the subject a pharmaceutical composition comprising an effective amount of a polynucleotide encoding an α9L9′T protein,
thereby protecting against or treating acoustic trauma in the subject.

2. (canceled)

3. The method of claim 1, wherein the subject:
(i) suffers from progressive hearing loss,
(ii) is exposed to noise or acoustic trauma,
(iii) is treated for a hearing-loss related pathology,
(iv) age-related hearing loss, or
(V) any combination thereof.

4. A method of protecting against or treating acoustic trauma in a subject comprising administering to the subject the pharmaceutical composition of claim 1, thereby protecting against or treating acoustic trauma in the subject.

5. The method of claim 4, wherein protecting against or treating acoustic trauma comprises, increasing sound evoked feedback inhibition, preventing noise-induced and/or preventing age-related hearing loss.

6. The method of claim 4, wherein administering comprises intracochlear injection, intratympanic injection, semicircular canal injection, round window injection, or a combination thereof.

7-9. (canceled)

10. The method of claim 5, wherein increasing sound evoked feedback inhibition, preventing noise-induced and/or preventing age-related hearing loss comprises mitigating noise-induced pattern of threshold shift.

11. The method of claim 10, wherein mitigating noise-induced pattern of threshold shift comprises reducing temporary or permanent auditory brainstem response (ABR) and distortion product otoacoustic emissions (DPOAEs) thresholds shifts and sensitivity to acoustic trauma.

12. The method of claim 11, wherein reducing temporary or permanent ABR threshold shifts comprises reducing maximal amplitude of ABR wave 1.

13. The method of claim 11, wherein reducing temporary or permanent ABR threshold shifts comprises decreasing ABR wave 1 latency shift.

14. The method of claim 11, wherein reducing temporary or permanent ABR threshold shifts comprises reducing or eliminating ABR shifts at all frequencies.

15. The method of claim 10, comprising limiting hearing loss to mid and upper frequencies.

16. The method of claim 4, further comprising administering or using other therapies to protect against or treat acoustic trauma.

17. The method of claim 16, wherein other therapies comprise ear covering, hearing aids, single gene repair, and/or small molecule therapy.

18. The method of claim 4, wherein the subject has noise-induced, age-related hearing loss, and/or a pathology exacerbated by noise.

19. (canceled)

20. A method of preventing noise-induced and/or age-related hearing loss, or increasing sound evoked feedback inhibition in a subject comprising administering to the subject a pharmaceutical composition comprising an effective amount of a polynucleotide encoding an α9L9′T protein,
thereby preventing noise-induced and/or age-related hearing loss or increasing sound evoked feedback inhibition in the subject.

21. The method of claim 1, wherein the polynucleotide is in a vector.

22-24. (canceled)

25. A vector comprising a polynucleotide encoding an α9L9′T protein.

26. A pharmaceutical composition comprising the vector of claim 25, and a pharmaceutically acceptable carrier.

27. The pharmaceutical composition of claim 26, wherein the pharmaceutically acceptable carrier is a gel or a nanoparticle.
